(19) United States
(12) Patent Application Publication
(10) Pub. No.: US 2019/0358432 A1
(43) Pub. Date: Nov. 28, 2019

(54) LOADING TOOLS FOR USE WITH A MEDICAL DEVICE

(71) Applicant: BOSTON SCIENTIFIC SCIMED, INC., MAPLE GROVE, MN (US)

(72) Inventors: JEFFRY D. JOHNSON, MAPLE GROVE, MN (US); MARY-CLAIRE ANDERSON, MINNEAPOLIS, MN (US); NICHOLAS LEE TASSONI, ANDOVER, MN (US); AJAY GUPTA, SHOREVIEW, MN (US); GARY JOHN PEDERSON, JR., ALBERTVILLE, MN (US)

(73) Assignee: BOSTON SCIENTIFIC SCIMED, INC., MAPLE GROVE, MN (US)

(21) Appl. No.: 16/420,880

(22) Filed: May 23, 2019

Related U.S. Application Data
(60) Provisional application No. 62/676,537, filed on May 25, 2018.

(51) Int. Cl. A61M 25/00; A61M 25/10

(52) U.S. Cl. CPC A61M 25/0082; A61F 2002/9522; A61M 25/10; A61M 25/005

(57) ABSTRACT

A medical device may be a loading tool including a tubular member having a lumen extending therethrough. The tubular member may include a distal portion and a proximal portion. The proximal portion of the tubular member may include a flexible member. The flexible member may be axially and radially adjustable between a first configuration and a second configuration. The distal portion of the tubular member may be configured to engage a valve, and the valve may be in communication with a lumen of an elongated medical device.

### CROSS-REFERENCE TO RELATED APPLICATIONS

This application claims the benefit of priority under 35 U.S.C. § 119 of U.S. Provisional Application No. 62/676,537, filed May 25, 2018, the entire disclosure of which is hereby incorporated by reference.

### TECHNICAL FIELD

The present disclosure pertains to medical devices, and methods for manufacturing medical devices. More particularly, the present disclosure pertains to loading tools for use with medical devices.

### BACKGROUND

A wide variety of intracorporeal medical devices have been developed for medical use, for example, intravascular use. Some of these devices include guidewires, catheters, loading tools, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. In a first aspect, a loading tool for use with a medical device may include a tubular member having a lumen extending therethrough. The tubular member may include a distal portion and a proximal portion. The proximal portion of the tubular member may include a flexible member. The flexible member may be axially and radially adjustable between a first configuration and a second configuration. The distal portion of the tubular member may be configured to engage a valve, and the valve may be in communication with a lumen of an elongated medical device.

In addition or alternative and in a second aspect, the distal portion of the tubular member may include an inner diameter and an outer diameter, the inner diameter may include a taper towards the distal end of the distal portion of the tubular member.

In addition or alternative and in a third aspect, the distal portion of the tubular member may include a distal tip portion configured to be received within the valve.

In addition or alternative and in a fourth aspect, the first configuration may be an expanded configuration and the second configuration may be a compressed configuration.

In addition or alternative and in a fifth aspect, the flexible member may be biased toward one of the first configuration and the second configuration.

In addition or alternative and in a sixth aspect, a proximal end of the distal portion may be coupled to a distal end of the proximal portion.

In addition or alternative and in a seventh aspect, the flexible member may include a flexible braid.

In addition or alternative and in an eighth aspect, the flexible member may include a flexible polymer.

In addition or alternative and in a ninth aspect, the flexible member may include a flexible braid and a flexible polymer layer may be disposed on the flexible braid.

In addition or alternative and in a tenth aspect, a loading tool for use with a medical device may include a tubular member. The tubular member may be configured to be disposed about a medical device. The tubular member may include a distal portion and a proximal portion. A distal end of the distal portion of the tubular member may be configured to engage an introducer device, and the proximal portion may be configured to be axially and radially adjustable between an expanded configuration and a compressed configuration.

In addition or alternative and in an eleventh aspect, the proximal portion of the tubular member may include a flexible member, the flexible member may include a polymer material.

In addition or alternative and in a twelfth aspect, the proximal portion of the tubular member may include a flexible member, the flexible member may include a metal braid.

In addition or alternative and in a thirteenth aspect, an inner diameter of a lumen of the proximal portion may be configured to adjust as the proximal portion adjusts between the expanded configuration and the compressed configuration.

In addition or alternative and in a fourteenth aspect, the proximal portion of the tubular member may be configured to return to the expanded configuration after being adjusted to the compressed configuration.

In addition or alternative and in a fifteenth aspect, an outer diameter of the distal portion may be configured to be received within an opening of the introducer device.

In addition or alternative and in a sixteenth aspect, a method of loading a medical device using a loading tool may include positioning a loading tool over a medical device. The loading tool may include a proximal portion and a distal portion, and at least a portion of the medical device may extend within the proximal portion of the loading tool. The method may include engaging the medical device through the proximal portion of the loading tool, and advancing the medical device through the loading tool by axially translating the proximal portion of the loading tool.

In addition or alternative and in a seventeenth aspect, the medical device may be a balloon catheter.

In addition or alternative and in an eighteenth aspect, engaging the medical device may include engaging a balloon portion of the balloon catheter.

In addition or alternative and in a nineteenth aspect, engaging the medical device through the proximal portion of the loading tool may include compressing the proximal portion of the loading tool.

In addition or alternative and in a twentieth aspect, the method may further comprise disengaging the medical device to allow the medical device to return to a biased configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic side view of an example loading tool in a first, expanded configuration;

FIG. 2 is a schematic cross-section view of the example loading tool of FIG. 1;

FIG. 3 is a schematic side view of an example loading tool in a second, compressed configuration;

FIG. 4 is a schematic side view of an example loading tool engaged with a valve and receiving a medical device; and

FIGS. 5A-5D are schematic cross-section views of a loading tool with a medical device inserted therein shown in a side view that depict an example method of using the loading tool.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Percutaneous angioplasty and the use of balloon catheters are common practices throughout the world. When using balloon catheters, a clinician may come into contact with the balloon. For example, when backloading a guidewire into the balloon catheter and/or when positioning the balloon catheter into packaging, a hemostasis valve, introducer, or other suitable component, the clinician may grasp or otherwise handle the balloon. For a number of reasons, it may be desirable to minimize contact with the balloon. For example, if the balloon includes a pharmacological coating or a stent with a pharmacological coating, handling the balloon could impact the coating, the stent, and/or the person handling the balloon.

Disclosed herein is a loading tool that helps reduce the amount of contact between a clinician and a medical device. Also disclosed are assemblies that help reduce contact with the medical device and methods for using (e.g., loading) medical devices, such as balloons, balloon catheters, and/or other medical devices.

In general, the loading tool may be configured to be used with a medical device such as a balloon catheter or other suitable medical device. Positioning the loading tool over the medical device as described herein may allow a clinician to “handle” the medical device without directly contacting the medical device. Similarly, other portions of a medical intervention such as guidewire loading/backloading, advancing the medical device through a hemostasis valve, introducer, and/or the like, withdrawing the medical device, and/or other processes may be completed while minimizing contact with the medical device. In addition to or as an alternative, the loading tool disclosed herein may be used for cardiac catheterization, stent delivery catheters, atherectomy devices, and/or any other suitable medical device.

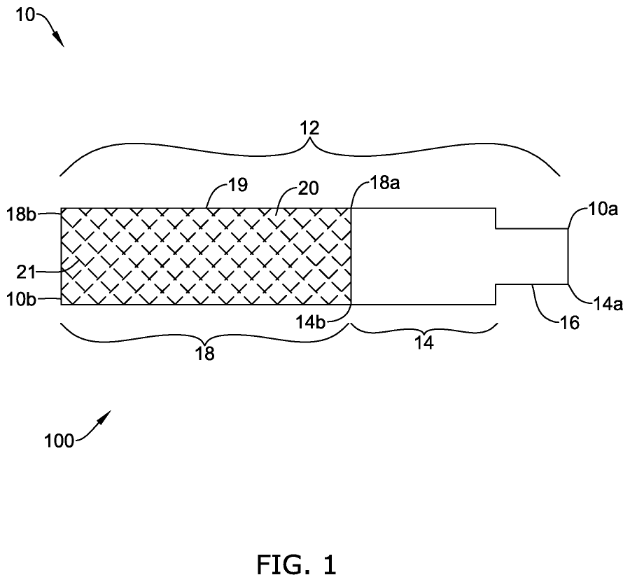

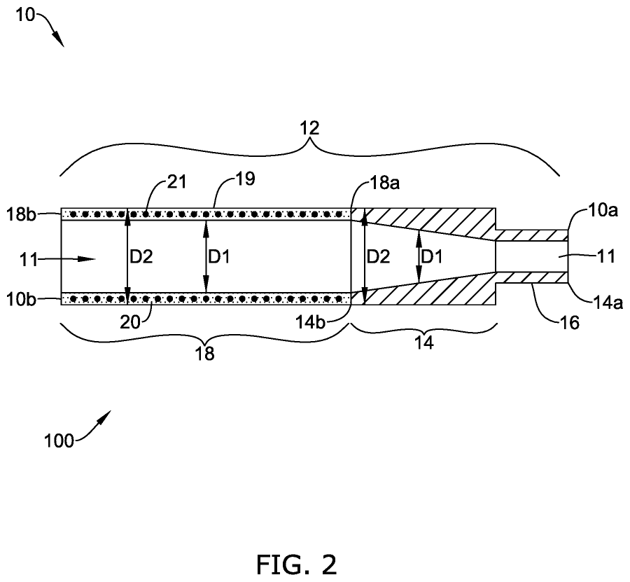

FIGS. 1 and 2 are illustrative of an example loading tool 10 in a first configuration 100 (e.g., an expanded configuration). FIG. 1 is a side view of the example loading tool 10 and FIG. 2 is a cross-section view of the example loading tool 10 of FIG. 1. The loading tool 10 may generally take the form of a tubular member 12 including a distal portion 14 having a distal end 14a and a proximal end 14b and a proximal portion 18 having a distal end 18a and a proximal end 18b. The loading tool 10 may include a first end 10a (e.g., a distal end, as shown in FIG. 1) and a second end 10b (e.g., a proximal end, as shown in FIG. 2), defining terminal ends of the loading tool 10. In some cases, the distal end 14a of the distal portion 14 may define the first end 10a, and the proximal end 18b of the proximal portion 18 may define the second end 10B, but this is not required.

As shown in FIG. 2, the loading tool 10 may include an inner diameter D1 defining a lumen 11 extending through the loading tool 10, and an outer diameter D2. The inner diameter D1 of the loading tool 10 may include a taper toward the distal end 14a of the distal portion 14. In some cases, where the inner diameter D1 of the loading tool 10 may taper, the outer diameter D2 of the loading tool 10 may remain constant at least proximate the distal portion 14. Alternatively, it may be contemplated that where the inner diameter D1 of the loading tool 10 may taper, the outer diameter D2 may also include a taper at least proximate the distal portion 14.

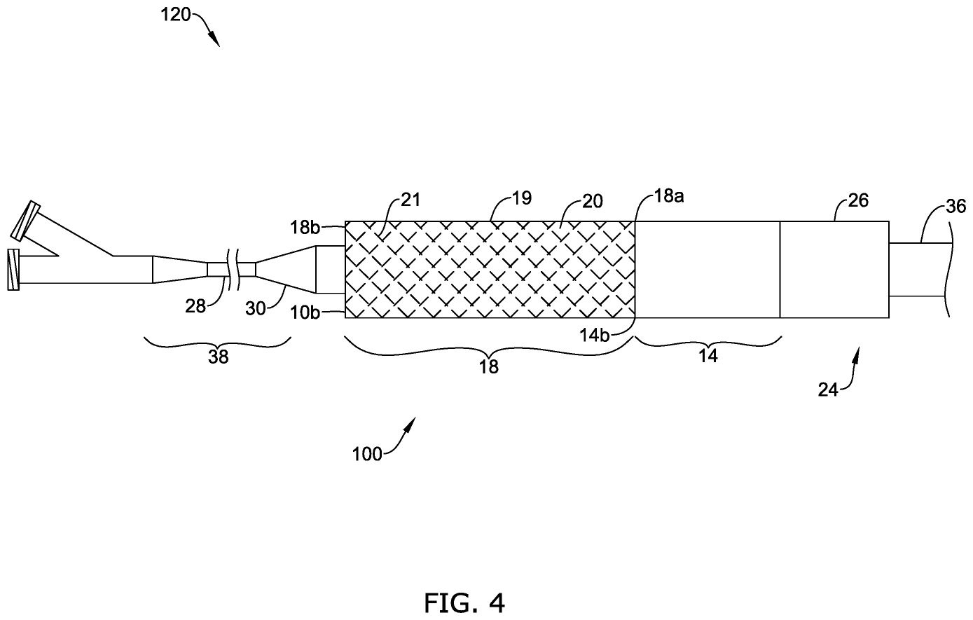

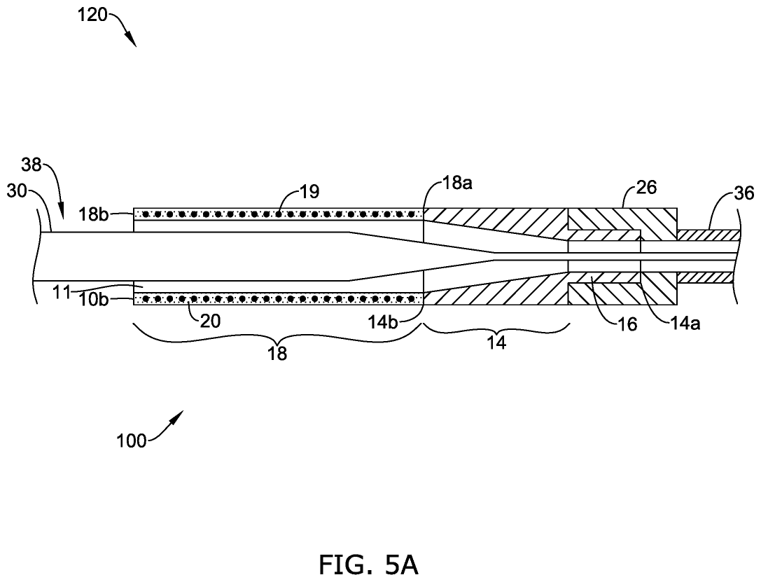

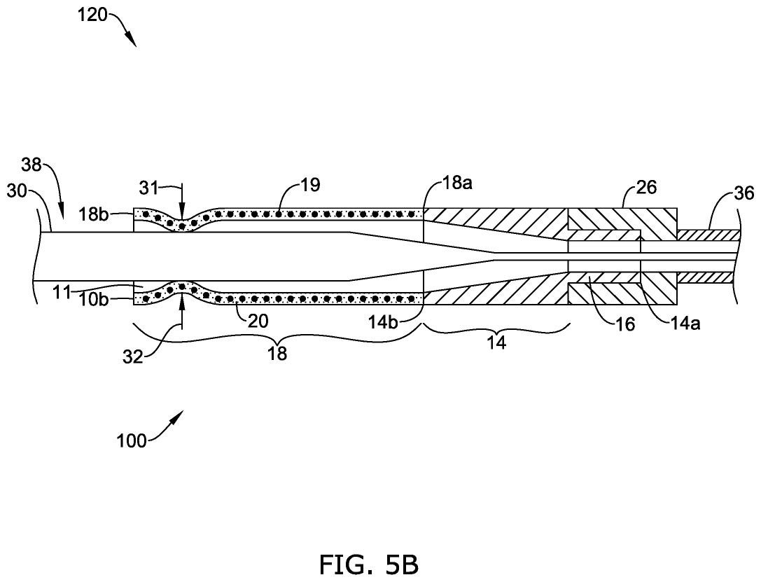

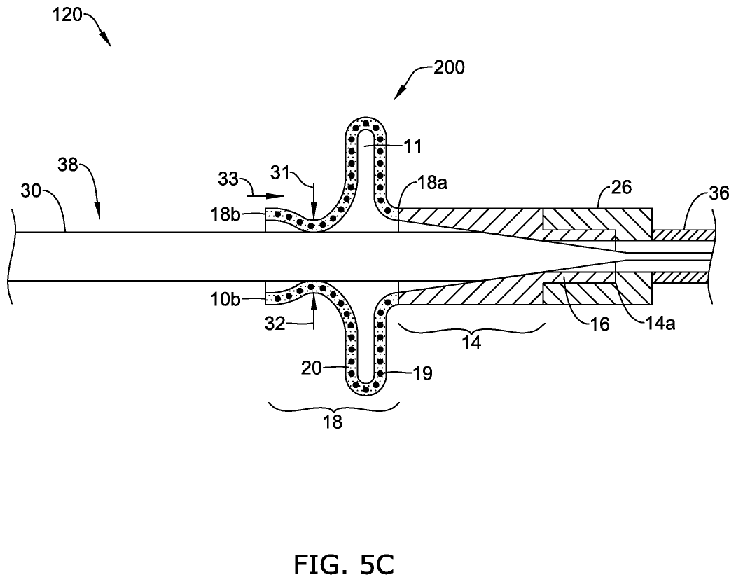

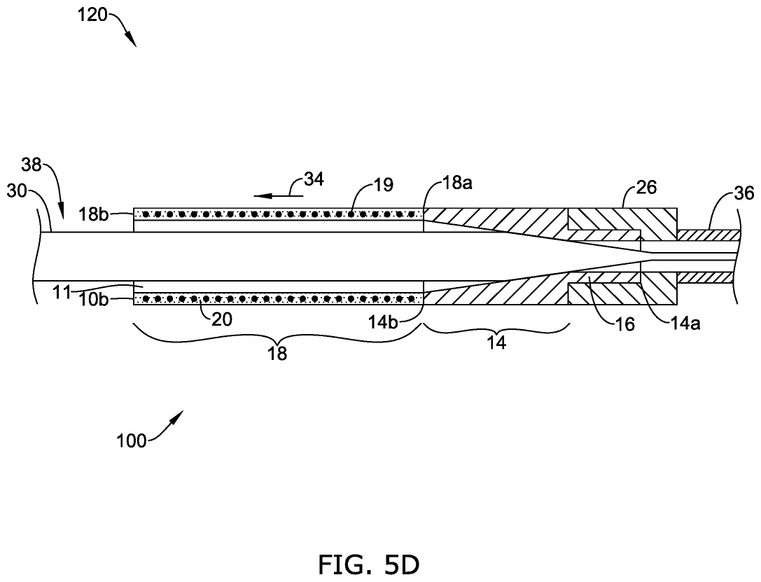

The distal portion 14 may include a distal tip 16 (e.g., a distal tip portion) extending to and/or at the distal end 14a of the distal portion 14. The distal tip 16 may have an outer diameter that is less than that of the proximal end 14b of the distal portion 14 and/or an outer diameter that is equal to or greater than that of the proximal end 14b of the distal portion 14. In some cases, the distal tip 16 may be configured to engage with a valve body (e.g., a hemostasis valve, a Touhy-Borst valve, or the like) of an introducer sheath or other suitable medical component, for example as shown in FIGS. 4-5D discussed below.

The distal tip 16 may have an outer diameter sufficient to engage with a valve body or other medical component and remain engaged with the valve body or medical component via a friction fit or other suitable fit. In some cases, the distal tip 16 may have an outer diameter of about 2.0 mm-5.0 mm, or about 2.8 mm-3.8 mm, or about 3.0 mm-3.8 mm, or other suitable size. As such, it is contemplated that the valve body, for example, may have an inner diameter of about 1.0 mm-5.0 mm, about 3.0 mm-4.3 mm, or about 3.3 mm-4.0 mm, or other suitable size. In one example, the distal tip 16 may have an outer diameter of about 3.0 mm and the inner diameter of the valve body may be sized and/or otherwise configured to engage the outer diameter of the distal tip 16 with a friction fit. In some cases, the distal tip 16 may include a ridge (not shown) to facilitate maintaining a connection between the distal tip 16 and the valve body and to mitigate the chances of the distal tip 16 from unintentionally disengaging the valve body.

The distal portion 14 of the tubular member 12 may have any suitable length. For example, the distal portion 14 may have a length of about 12 mm-50 mm, about 18 mm-38 mm, or about 20-30 mm. In one example, the distal portion 14 may have a length of about 25 mm.

The distal portion 14 of the tubular member 12 may be formed from a relatively stiffer or more rigid material than the proximal portion 18 (discussed below). For example, the distal portion 14 may be formed of a rigid plastic, stainless steel hypotube, and/or other suitable materials.

When the distal portion 14 is formed from a rigid plastic, stainless steel hypotube and/or the like, the inner diameter D1 of the loading tool 10 may be prevented from changing shape or decreasing in diameter, when a radially inward force is applied to an external surface of the distal portion 14. As such, in some embodiments, the distal portion 14 may be configured to provide structural support when advancing the loading tool 10 into a valve body, introducer, dilator or the like. For example, when advancing the loading tool 10 into a hemostasis valve, the valve may exert some radially inward pressure (e.g., compression) onto the loading tool 10 and the structural support provided by the distal portion 14 may reduce an amount of pressure/force that could be transferred to, for example, a balloon, balloon catheter, and/or other medical device extending through the loading tool 10. Similarly, the distal portion 14 may allow for a user to hold or grip the loading tool 10 without transferring a force caused by holding or gripping the distal portion 14 to a balloon catheter or other suitable medical device within the loading tool 10.

In at least some embodiments, the proximal end 14b of the distal portion may be coupled to the distal end 18a of the proximal portion 18 of the tubular member 12. The distal portion 14 of the tubular member 12 may be coupled to the proximal portion 18 of the tubular member 12 by heat molding, adhesive bonding, insert molding, and/or other suitable connecting techniques. Alternatively, the distal portion 14 and proximal portion 18 of the tubular member 12 may be formed from one continuous structure.

The proximal portion 18 of the tubular member 12 may have any suitable length. For example, the proximal portion 18 may have a length of about 25-127 mm, or about 25-102 mm, or about 50-76 mm. In one example, the proximal portion 18 of the tubular member 12 may have a length of about 25.4 mm. In addition or alternatively, the proximal portion 18 of the loading tool 10 may have an original, expanded length that can cover at least a portion of a balloon on a balloon catheter (e.g., a balloon having a length of about 200 mm or greater, 200 mm or less, 150 mm or less, 100 mm or less, or another suitable length).

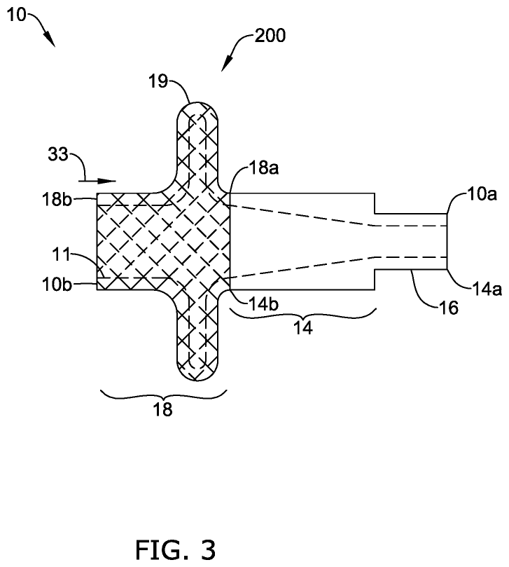

The proximal portion 18 of the tubular member 12 may be flexible such that the proximal portion 18 may be axially and/or radially adjustable between the first configuration 100 and a second configuration 200 (as shown in FIG. 3). To facilitate axially and/or radially adjusting the proximal portion 18 and/or for other suitable structural or non-structural purposes, the proximal portion 18 may include a flexible member 19. Example configurations of the flexible member 19 may include, but are not limited to, a flexible braid, a braid, a helical spring, a spring, a flexible polymer structure, a cut tube, and/or the like. The material of the flexible member 19 may be configured to bias the proximal portion 18 in a certain configuration (e.g., the first configuration 100, and/or the second configuration 200 (e.g., as shown in FIG. 3), and/or any other desirable configuration).

The flexible member 19 may be formed from a shape memory material, such as a flexible metal (e.g., nickel-titanium alloy and/or other suitable metal), a flexible polymer, and/or any other suitable material to allow for axial and radial adjustment of the proximal portion 18 and a bias to a desired configuration. In some examples, when the flexible member 19 of the tubular member 12 includes a structural component 21 (e.g., a flexible braid, spring, cut tube, or other suitable structural component), the flexible member 19 may optionally also include a flexible polymer material (e.g., polyolefin, nylon, polypropylene and/or any other suitable material) disposed on and/or about the flexible member 19. As shown in FIGS. 1 and 2, the flexible polymer material may form a flexible polymer layer 20 over and/or around the structural component 21 (e.g., the flexible braid, the spring, cut tube, or other suitable flexible structure). The flexible polymer material may be disposed on and/or about the structural component 21 by heat shrink, molding, adhesive bonding, insert molding, and/or other suitable technique. The structural component 21 is shown in broken lines in FIG. 1 due to encapsulation within the flexible polymer layer 20.

The flexible polymer layer 20, when included, may provide the proximal portion 18 with an inner surface that will not interfere with a medical device (e.g., a balloon of a balloon catheter having a pharmaceutical coating) inserted into the loading tool 10. Additionally or alternatively, the flexible polymer layer 20 may provide a barrier between the balloon and the clinician, thereby allowing the clinician to “handle” the balloon, and facilitating the clinician gripping the balloon or other medical device.

When the proximal portion 18 of the tubular member 12 is in the first configuration 100, the lumen 11 of the tubular member 12 may have a constant inner diameter D1 at least proximate the proximal portion 18, but this isn't always required. The inner diameter D1 of the proximal portion 18 of the tubular member 12 may be sufficient to allow, for example, a balloon of a balloon catheter to pass therethrough (e.g., a balloon having an outer diameter of about 2.1 mm, or about 2.3 mm, or any other suitable size).

FIG. 3 is a side view of the example loading tool 10 where the proximal portion 18 of the tubular member 12 is in the second configuration 200 (e.g., a compressed configuration). The lumen 11 (shown as defined by broken lines in FIG. 3) of the loading tool 10 may be configured to adjust (e.g., axially and/or radially) as the proximal portion 18 adjusts between the first configuration 100 (shown in FIGS. 1 and 2) and the second configuration 200 (e.g., adjusts in a direction indicated by arrow 33 or in a direction opposite of the direction indicated by the arrow 33), at least proximate the proximal portion 18. For example, when the proximal portion 18 of the tubular member 12 is in the second configuration 200, the lumen 11 of the loading tool 10 may have an inner diameter in at least one location that is greater than the inner diameter of the lumen 11 when the proximal portion 18 is in the first configuration 100. Additionally or alternatively, when the proximal portion 18 is in the second configuration 200, as shown in FIG. 3, the length of the proximal portion 18 may be less than that of the length of the proximal portion 18 when in the first configuration 100.

In some cases, the proximal portion 18 of the tubular member 12 may be biased toward the first configuration 100. In other cases, the proximal portion 18 of the tubular member 12 may be biased toward the second configuration 200.

An extent to which the proximal portion 18 of the tubular member 12 may compress may vary as the proximal portion adjusts between the first configuration 100 and the second configuration. For example, the proximal portion 18 may compress to a length that is about fifty (50) percent or less of the “original” (e.g., “expanded”) length, or about forty (40) percent or less of the original length, or about thirty (30) percent or less of the original length, or about twenty (20) percent or less of the original length, or about ten (10) percent or less of the original length. These are just examples.

FIG. 4 illustrates a medical device assembly 120, including the loading tool 10, a balloon catheter 38 (e.g., a medical device received within the loading tool 10), and an introducer 36. The balloon catheter 38 may include a catheter shaft 28 and a balloon 30 attached to the catheter shaft 28. In at least some embodiments, the balloon 30 may include a pharmaceutical coating, and/or a stent or endoprosthesis disposed thereon (not shown). The stent or endoprosthesis may include a pharmaceutical coating. The balloon 30 may be configured to shift between a generally collapsed state (where the balloon 30 may be in a folded configuration and have one or more wings or folds formed therein) and an expanded configuration. In FIG. 4, the balloon 30 is shown schematically in a collapsed state.

In use, the loading tool 10 may be disposed about the catheter shaft 28. This may include positioning the balloon 30 proximal of the loading tool 10. Alternatively, the loading tool 10 may be disposed about a portion of the balloon 30, so at least a portion of the balloon 30 extends within the proximal portion 18 of the loading tool 10, as shown in FIG. 4. When suitably configured, the catheter shaft 28 including the balloon 30, and the loading tool 10 may be loaded into or otherwise advanced into a suitable introducer, dilator, or the like (e.g., an introducer sheath 24) as shown in FIG. 4, and ultimately into a body lumen. This may include inserting (e.g., via gripping, compressing, friction fitting, and/or any other suitable method) the balloon 30 through the proximal portion 18 of the loading tool 10 and inserting the loading tool 10 into the valve body 26 (e.g., a hemostasis valve, a touhy-borst valve, or the like). Axial translation of the proximal portion 18 of the loading tool 10 in a distal direction may adjust the proximal portion 18 from the first configuration 100 (as shown in FIGS. 1 and 2) to the second configuration 200 (as shown in FIG. 3) to facilitate inserting the balloon catheter 38 into the introducer sheath 24, as discussed in greater detail below. Disengaging the proximal portion 18 of the loading tool 10 may allow the proximal portion 18 to return to the first configuration 100 either manually or automatically (e.g., in response to bias force).

FIGS. 5 A-5D are cross-section views of the loading tool 10 used in an exemplary method of use for the loading tool 10 when the proximal portion 18 is biased toward the first configuration 100. As shown in FIG. 5A, the proximal portion 18 of the loading tool 10 may be biased toward and/or positioned in the first configuration 100 (e.g., the expanded configuration). Advancing the balloon 30 may include engaging (e.g., via gripping, compressing, friction fitting, and/or any other suitable method) the balloon 30 through the proximal portion 18 of the loading tool 10, as shown in FIG. 5B (indicated by arrows 31 and 32), and axially translating (e.g., via pushing, sliding, or any other suitable method) the proximal portion 18 of the loading tool 10 in a distal direction, as shown in FIG. 5C (indicated by the arrow 33). Axial translation of the proximal portion 18 of the loading tool 10 in a distal direction while engaging the balloon 30 through the proximal portion 18 may adjust the proximal portion 18 from the first configuration 100 to the second configuration 200, and advance the balloon catheter 38 into the introducer 36. After the balloon catheter 38 has been advanced, the proximal portion 18 of the loading tool 10 may be disengaged to allow the proximal portion 18 to return to the first configuration 100, as shown in FIG. 5D (indicated by arrow 34). The steps of engaging the balloon 30 through the proximal portion 18, axially translating the proximal portion 18, and disengaging the proximal portion 18, may be repeated as necessary until the balloon catheter 38 has reached a desired location.

Although the method depicted in FIGS. 5A-5D utilizes a balloon catheter 38, the method may be utilized to advance other elongated medical devices into an introducer, sheath, or body lumen. Further, it is contemplated that the disclosed steps may be performed in one or more other orders and/or one or more steps may be included before, in between, and/or after the disclosed steps. Yet further, the loading tool 10 may be utilized for one or more other methods of loading or unloading a medical device from another component.

While not intending to be limiting, a variety of different dimensions are contemplated for the loading tool 10. Some of the dimensions that are contemplated are disclosed herein. The loading tool 10 may have a length of about 20-300 mm, or about 100-200 mm, or about 125-175 mm, or other suitable length. In general, the loading tool 10 may have a length suitable to contain at least a portion of a medical device (e.g., a balloon catheter or a balloon of a balloon catheter) therein. For example, the proximal portion 18 may have a size of about 25-127 mm and the distal portion 14 may have a size of up to around 25 mm.

The materials that can be used for the various components of the loading tool 10 and the various tubular members disclosed herein may include those commonly associated with medical devices. For simplicity purposes, the following discussion makes reference to the loading tool 10. However, this is not intended to limit the devices and methods described herein, as discussion may be applied to other similar tubular members and/or components of tubular members or devices disclosed herein.

Loading tool 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of loading tool 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of loading tool 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of loading tool 10 to achieve the same result.

In some embodiments, a degree of Magnetic Resonance Imaging (Mill) compatibility is imparted into loading tool 10. For example, loading tool 10, or portions thereof, may be made of a material that does not substantially distort the image and create substantial artifacts (i.e., gaps in the image). Certain ferromagnetic materials, for example, may not be suitable because they may create artifacts in an MRI image. Loading tool 10, or portions thereof, may also be made from a material that the Mill machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A loading tool for use with a medical device, the loading tool comprising:
a tubular member having a lumen extending therethrough, the tubular member including a distal portion and a proximal portion;
wherein the proximal portion includes a flexible member, the flexible member is axially and radially adjustable between a first configuration and a second configuration; and
wherein the distal portion is configured to engage a valve in communication with a lumen of a medical device.

2. The loading tool of claim 1, wherein the distal portion of the tubular member has an inner diameter and an outer diameter, the inner diameter includes a taper toward a distal end of the distal portion of the tubular member.

3. The loading tool of claim 1, wherein the distal portion of the tubular member includes a distal tip portion configured to be at least partially received within the valve.

4. The loading tool of claim 1, wherein the first configuration is an expanded configuration and the second configuration is a compressed configuration.

5. The loading tool of claim 4, wherein the flexible member is biased toward one of the first configuration and the second configuration.

6. The loading tool of claim 1, wherein a proximal end of the distal portion is coupled to a distal end of the proximal portion.

7. The loading tool of claim 1, wherein the flexible member comprises a flexible braid.

8. The loading tool of claim 1, wherein the flexible member comprises a flexible polymer.

9. The loading tool of claim 1, wherein the flexible member comprises a flexible braid and a flexible polymer material disposed on the flexible braid.

10. A loading tool for use with a medical device, the loading tool comprising:
a tubular member configured to be disposed about a medical device, the tubular member including a distal portion and a proximal portion;
wherein a distal end of the distal portion is configured to engage an introducer device; and
wherein the proximal portion is configured to be axially and radially adjustable between an expanded configuration and a compressed configuration.

11. The loading tool of claim 10, wherein the proximal portion of the tubular member includes a flexible member, the flexible member comprising a polymer material.

12. The loading tool of claim 10, wherein the proximal portion of the tubular member includes a flexible member, the flexible member comprising a metal braid.

13. The loading tool of claim 10, wherein an inner diameter of a lumen of the proximal portion is configured to adjust as the proximal portion adjusts between the expanded configuration and the compressed configuration.

14. The loading tool of claim 10, wherein the proximal portion of the tubular member is configured to return to the expanded configuration after being adjusted to the compressed configuration.

15. The loading tool of claim 10, wherein an outer diameter of the distal portion is configured to be received within an opening of the introducer device.

16. A method of loading a medical device using a loading tool, the method comprising:
positioning a loading tool over a medical device, the loading tool having a proximal portion and a distal portion with at least a portion of the medical device extending within the proximal portion of the loading tool;
engaging the medical device through the proximal portion of the loading tool; and
advancing the medical device through the loading tool by axially translating the proximal portion of the loading tool while engaging the medical device through the proximal portion of the loading tool.

17. The method of claim 16, wherein the medical device is a balloon catheter.

18. The method of claim 17, wherein engaging the medical device includes engaging a balloon portion of the balloon catheter.

19. The method of claim 16, wherein engaging the medical device through the proximal portion of the loading tool includes compressing the proximal portion of the loading tool.

20. The method of claim 16 further comprising:
disengaging the medical device to allow the medical device to return to a biased configuration.
